(19) United States
(12) Patent Application Publication
(10) Pub. No.: US 2013/0327663 A1
(43) Pub. Date: Dec. 12, 2013

(54) Surgical Instrument Holder

(71) Applicant: Brett Telford, Millswood (AU)

(72) Inventor: Brett Telford, Millswood (AU)

(21) Appl. No.: 13/915,118

(22) Filed: Jun. 11, 2013

Related U.S. Application Data
(60) Provisional application No. 61/689,722, filed on Jun. 11, 2012.

(51) Int. Cl. A61B 19/10; B65D 21/02; A61B 19/02

(52) U.S. Cl. CPC A61B 19/10; A61B 19/0256; B65D 21/0209
USPC 206/363; 128/852; 220/23.4

(57) ABSTRACT

A surgical instrument holder that can be affixed to a surgical drape and provide a receptacle for holding surgical instrument during an operation. The surgical instrument holder has an elongated hollow body with an outwardly flared top to facilitate inserting an instrument therein. A connection system allows the user to couple a plurality of surgical instrument holders together to facilitate the holding of multiple instruments.

### RELATED APPLICATIONS

The present patent application is based upon and therefore claims priority to Provisional Patent Application Ser. No. 61/689,722, filed Jun. 11, 2012 and entitled “Surgical Instrument Holder”.

### FIELD OF THE INVENTION

The present invention relates to a holder for retaining instruments, and, more particularly, to a holder that is used to retain a surgical instrument or instruments during an operation at a location convenient to the medical personnel, such as a surgeon.

### BACKGROUND OF THE INVENTION

In many surgical operation, the surgeon utilizes different instruments in carrying out the operation and, at times, the surgeon substitutes one instrument for another during the operation to carry out a different function or procedure on the patient.

One problem, however, with that process, is that the surgeon needs some place to rest each instrument that is not currently in use in a clean environment and that location must also be convenient to the surgeon so that the instrument can again be used if needed. Accordingly, there must be ready access to each instrument by the surgeon.

The problem is, of course, that the surgeon, scrub assistant or other attending medical personnel can lay down an instrument when it is not currently being used in a location that. is unstable and the instrument can fall to the floor, thereby requiring re-sterilization or the use of an alternate instrument. In any case, the need to re-sterilize or provide an alternate instrument is an inconvenience to the surgeon and may interrupt the smooth flow of the operation and create a distracting annoyance to the surgeon who is concentrating on the procedures being undertaken.

Also, certain instruments, such as electrocautery instruments, that are used in medical procedures are at an extreme temperature and which could be harmful to the patient, surgical personnel or materials in the sterile field if the high temperature instrument would inadvertently conic in contact with a person or sensitive material. As such, it is important for such high temperature instruments to be located readily in a position or location where the threat of contacting some adverse person or material is eliminated, or at least drastically reduced, to reduce the potential for burns and fire.

Accordingly, it would be advantageous to have an instrument holder that can be attached, for example, to the surgical drape covering the patient and thus be convenient to the surgeon and yet provide a safe place for the surgeon to place an instrument in a clean environment where the instrument can still be readily accessed for later use.

### SUMMARY OF THE INVENTION

in accordance with the present invention, there a surgical instrument holder that is made up of an elongated, hollow body. The elongate hollow body has an upper end and a lower end and, preferably, the upper end tapers outwardly to accommodate the insertion of a surgical instrument therein. An opening is provided in the elongated, hollow body for use in attaching the surgical instrument holder to a surgical drape by conventional means such as a towel clamp.

In an exemplary embodiment, there is a connection system such that multiple surgical instrument holders can be readily affixed together by a snap action and can also be easily detached from each other at the desire of the surgeon. In one embodiment, the connection system includes a cylindrical member on the surgical instrument holder and a set of clips about 180 degrees from the cylindrical member such that the cylindrical member of one surgical instrument holder can simply snap into and thereby become affixed to the clips of an adjacent surgical. instrument holder,

The surgical instrument holder can be constructed of a material that disintegrates at an elevated temperature such that the surgical instrument holder is limited to a single use and any attempt to autoclave the surgical instrument holder will result in its destruction. On the other hand, the present surgical instrument holder can be made reusable and thus constructed of strong material including, but not limited to, metal.

These and other features and advantages of the present invention will become more readily apparent during the following detailed description taken in conjunction with the drawings herein,

### BRIEF DESCRIPTION OF THE DRAWINGS

FIG. 1 is a perspective view of a surgical instrument holder constructed in accordance. with the present invention;

FIG. 2 is a perspective view of a plurality of surgical instrument holders of the present invention affixed together; and

FIG. 3 is a perspective view of various representative surgical instruments contained within surgical instrument holders of the present invention.

### DETAILED DESCRIPTION OF THE INVENTION

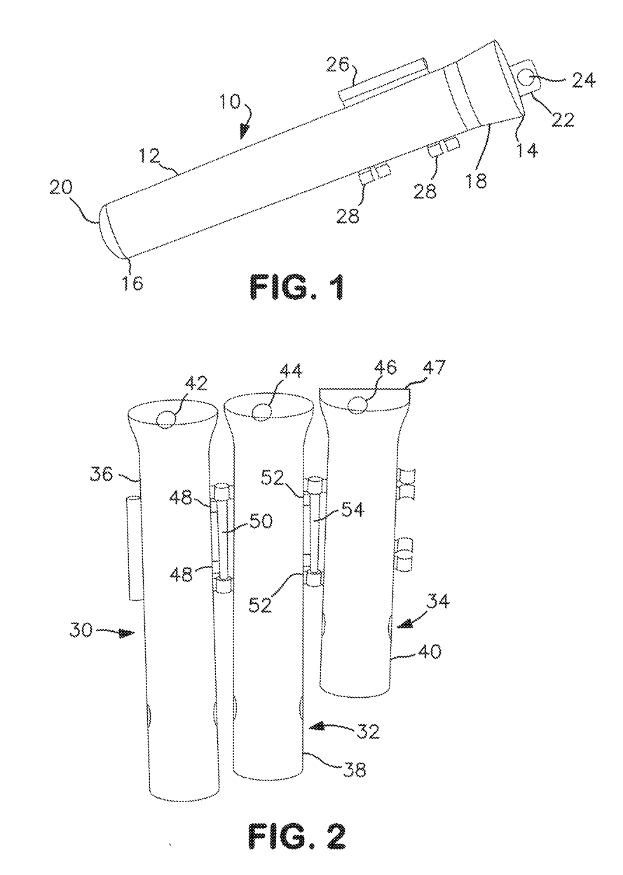

Turning first to FIG. 1, there is shown a perspective view of a surgical instrument holder 10 constructed in accordance with the present invention. As can be seen, the surgical instrument holder 10 is comprised of a hollow, elongated body 12. In the exemplary embodiment of FIG. 1, the hollow, elongated body 12 is cylindrical, having a circular, lateral cross section, however, as will become clear, the lateral cross section may be of other geometric shapes such as triangular, square and the like and including a shape wherein at least a portion of the body 12 has a flat, planar surface.

The surgical instrument holder 10 has an upper end 14 and a lower end 16 and, as is conventional, the upper and lower ends 14, 16 refer to the orientation and location of those ends when the surgical instrument holder 10 is in its operative position. Proximate to the upper end 14, there is an outwardly tapered portion 18 that allows the particular surgical instrument to be more easily inserted into the upper end 14 of the surgical instrument holder 10.

The lower end 16 may be closed with a bottom 20 or may remain open. As also can be seen in FIG. 1, there is a tab 22 with an opening 24 to allow the surgical instrument holder 10 to be firmly affixed to a surgical drape that covers the operating table. With the opening 24, a conventional towel clamp (not shown) can be used to carry out the securement such that the surgical instrument holder 10 is orientated with the upper end 14 in the upward direction and the lower end 16 generally vertically aligned below the upper end 14 so that a surgical instrument can easily be inserted through the upper end 14 and remain in the surgical instrument holder 10.

In FIG. 1, there is also a connection system that allows one surgical instrument holder 10 to be affixed to another, adjacent surgical instrument holder 10. While a variety of connection systems may be used to secure adjacent surgical instrument holder 10 together, one means, as shown in FIG. 1 is with the use of a cylindrical member 26 affixed to the hollow, elongated body 12 to extend outwardly therefrom and a set of arced clips 28 about 180 degrees displaced from the cylindrical member 26 such that the cylindrical member 26 is dimensioned to snap lit into and be retained by the clips 28.

Turning then to FIG. 2 there is shown a perspective view of a plurality of surgical instrument holders 30, 32, 34, each comprising a hollow, elongated body 36, 38, 40. In this embodiment, instead of an opening 24 formed in a tab 2.2 (FIG. 1), there are openings 42, 44, 46 actually provided in the hollow, elongated bodies 36, 38, 40. As can also be seen, the surgical instrument holders 30, 32 and 34 are secured together by means of the connection system of FIG. 1, that is, the clips 48 of surgical instrument holder 30 are securely snapped onto the cylindrical member 50 of the surgical instrument holder 32 to affix those surgical instrument holders together.

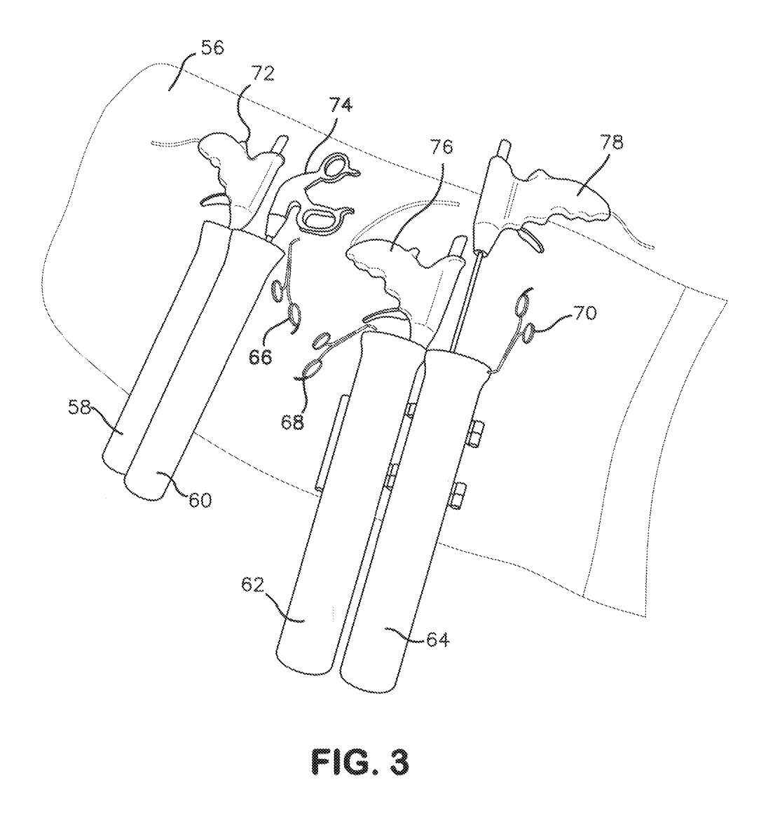

As also can be seen in FIG. 3, the surgical instrument holder 34 has a flat surface 47, that is oriented so as to be facing the patient (posterior) and lay against a surgical drape. With the flat surface 47 facing the patient, once attached to the surgical drape of other material, the surgical instrument holder has a reduced tendency to roll and, if a plurality of side by side surgical instrument holders are used, all with the flat surface facing the patient, the inadvertent separation of the surgical instrument holders is prevented. The use of the flat surface 47 is shown on only one of the surgical instrument holders 30, 32, 34 in FIG. 2, however, it can be seen that all of the surgical instrument holder can have the flat surface.

The flat surface 47 may be fully along the vertical length of a surgical instrument holder or at least substantially.

In a similar manner, the clips 52 of the surgical instrument holder 32 are snap fitted to the cylindrical member 54 of the surgical instrument holder 40. As such, all three of the surgical instrument holders 30, 32 and 34 are affixed together in a snap lit so that the surgeon can have more than one surgical instrument holder to hold multiple surgical instruments or unsnap them and resort to the use of only one such surgical instrument holder.

As a further convenience, the length of each of the surgical instrument holders 30, 32, 34 can, as shown, be different so as to accommodate different size and/or lengths of surgical instruments.

Turning finally to FIG. 3, there is shown a perspective view of a surgical drape 56 that normally is used to cover the patient and a plurality of surgical instrument holders 58, 60, 562 and 64 are shown hanging from that surgical drape 56 in order to be close at hand to the surgeon. The surgical instrument holders 58, 60, 62, 64 may be secured to the surgical drape 567 in a variety of manners, however, in the exemplary embodiment of FIG. 3, there are towel clamps 66, 68, and 70 that are used to clamp the surgical instrument holders 58, 60, 62, 64 onto the surgical drape 56 after the surgical drape 56 has passed through one of the openings described in FIGS. 1 and 2.

As other embodiments, the surgical instrument holder can be constructed of a material or materials that are flame proof to prevent burns or fires in surgical operating room. In addition, the surgical instrument holder can be constructed of a resorbable material that is environmentally friendly. Additionally, the material of construction may be a molded plastic construction or a material that is degradable and will disintegrate during the normal autoclave or sterilization in order to limit the use of the surgical instrument holder to a single use, the eliminating the potential of cross contamination.

As a still further embodiment, the present surgical instrument holder can be a part of a kit that is comprised of single use surgical tools or instruments and the surgical instrument holder can be packaged with the single use tools or instruments.

Those skilled in the art will readily recognize numerous adaptations and modifications which can be made to the surgical instrument holder and method of constructing the same of the present invention which will result in an improved surgical instrument holder and method, yet all of which will fall within the scope and spirit of the present invention as defined in the following claims. Accordingly, the invention is to he limited only by the following claims and their equivalents.

What is clamed is:

1. A surgical instrument holder comprising an elongated, hollow body having an upper, open end and a lower end, the upper end tapering outwardly, an opening, for attaching the surgical it holder to a surgical drape, and a connection system that enables one surgical instrument holder to be affixed to another surgical instrument holder.

2. The surgical instrument holder of claim 1 wherein the connection system comprises a cylindrical member on one surgical instrument holder that interlits into clips provided on an adjacent surgical instrument holder

3. The surgical instrument holder of claim 1 wherein the elongated hollow body is comprised of a material that disintegrates at elevated temperatures.

4. The surgical instrument holder of claim 1 wherein the elongated, hollow body is perforated for drainage and cooling.

5. The surgical instrument holder of claim 1 wherein the elongate, hollow body is comprised of a reusable metallic material.

6. The surgical instrument holder of claim 1 wherein the surgical instrument holder has an elongate hollow cylindrical body.

7. The surgical instrument holder of claim 1 that is constructed of a material that is flame proof to prevent burns or fires in a surgical operating room.

8. The surgical instrument holder of claim 1 wherein the surgical instrument holder is constructed of a resorbable material that is environmentally friendly.

9. The surgical instrument holder of claim 1 wherein the surgical instrument holder has a fiat surface at least substantially along a vertical length thereof.

10. The surgical instrument holder of claim 9 wherein the surgical instrument holder has a flat surface that is fully along a vertical length thereof.

11. A kit comprised of a plurality of the surgical instrument holders of claim 1 wherein the surgical instrument holders are different dimensionally.

12. The kit of claim 11 wherein the surgical tools or instruments are single use tools or instrument.

13. The kit of claim 11 wherein at least two surgical instrument holders in the kit have a connection system to allow a plurality of surgical instrument holders to be affixed together.

14. A surgical environment, a surgical drape that is positioned atop of a patient, at least one surgical instrument holder affixed to the surgical drape, the surgical instrument holder comprised of an elongated, hollow body having an upper, open end and a lower end, the upper end tapering outwardly and an opening for attaching the surgical instrument holder to a surgical drape, and as connection system that enables one surgical instrument holder to be affixed to another surgical instrument holder.

15. The surgical environment of claim 14 wherein the surgical instrument holder has a connection system that enables one surgical instrument holder to be affixed to another surgical instrument holder.

16. The surgical environment of claim 14 wherein the at least one surgical instrument holder comprises a plurality of surgical instrument holders affixed to each other.

17. The surgical environment of claim 15 wherein the connection system provides a snap it to affix surgical instrument holders to each other.

18. The surgical environment of claim 15 wherein the connection system comprises a cylindrical member on one surgical instrument holder that interfits into clips provided on an adjacent surgical instrument holder

19. The surgical environment of claim 14 wherein the least one surgical instrument holder has a fiat surface facing a patient.

20. The surgical environment of claim 16 wherein each of the plurality of surgical instrument holders has a flat surface facing a patient
